(19) United States
(12) Patent Application Publication
(10) Pub. No.: US 2007/0072287 A1
(43) Pub. Date: Mar. 29, 2007

(54) Biomems cartridges

(75) Inventors: Dallas T. Morisette, Lafayette, IN (US); Kevin F. Lee, East Greenwich, RI (US); Hany Selim, Sunnyvale, CA (US); Bahadir Erimli, Campbell, CA (US); Laila R. Razouk, Sunnyvale, CA (US); Rashid Bashir, West Lafayette, IN (US)

Correspondence Address: COOLEY GODWARD KRONISH LLP, 3000 EL CAMINO REAL, 5 PALO ALTO SQUARE, PALO ALTO, CA, 94306, US

(73) Assignee: BioVitesse, Inc., San Jose, CA (US)

(21) Appl. No.: 11/440,432

(22) Filed: May 23, 2006

Related U.S. Application Data
(60) Provisional application No. 60/683,750, filed on May 23, 2005.

(51) Int. Cl. C12M 1/34; C12Q 1/68

(52) U.S. Cl. 435/287.2; 435/6

(57) ABSTRACT

A cartridge includes a first layer to receive a printed circuit board hosting a biochip. A second layer is connected to the first layer and is configured for sample injection to the biochip. The second layer includes a first septum to isolate fluidic ports. A third layer is connected to the second layer and is configured for fluidic purge operations. The third layer includes a second septum to isolate a fluidic channel. A fourth layer is connected to the third layer and is configured to receive and route fluidic samples to the third layer.

### CROSS REFERENCE TO RELATED APPLICATIONS

This application claims the benefit of U.S. Provisional Application No. 60/683,750, filed May 23, 2005, entitled “Techniques and Devices to Interface Macroscale Samples to Micro and Nanodevices”, the contents of which are incorporated herein by reference.

### BRIEF DESCRIPTION OF THE INVENTION

This invention relates generally to Biological Micro-Electro-Mechanical systems (BioMEMS) and Biological Integrated Nanosystems. More particularly, this invention relates to cartridges for interfacing macro-scale fluid samples to BioMEMS chips and integrated chips with micro and nanoscale sensors.

### BACKGROUND OF THE INVENTION

Micro-Electro-Mechanical Systems (MEMS) are well known. BioMEMS is a branch of MEMS that evolved in the past decade. BioMEMS have gained much attention from the research community in an effort to facilitate biomedical applications. In general, BioMEMS contemplates all disciplines related to life sciences integrated to micro- or nanoscale systems. As used herein, the term BioMEMS or biochip refers to any micro- or nanoscale system configured for a life sciences application. Research seeks ways to improve diagnostics, drug discovery, drug delivery, tissue engineering, and therapeutics methods. Devices for the characterization and detection of DNA and protein are prime examples of BioMEMS application in biology and medicine. Point-of-Care (POC) systems that utilize BioMEMS are useful in diagnostic environments. Implantable BioMEMS is another kind of innovative device that functions as a drug delivery system or therapeutic treatments for disorders, such as paralysis or Parkinson's disease.

Microfluidics is a vital component to the success of BioMEMS due to the natural phenomena effects that occur at small scales. Depending on the materials used in BioMEMS, the traditional assumption of continuum flow regime may not be valid if the length scale is small. Molecular interaction, such as Brownian motion, may have to be considered. Other new effects imposed by microfluidic forces, such as changes of electrical properties, chemical reactions, and cellular activities, are of interest to a multidisciplinary audience from various fields of engineering to life science to chemistry.

Micro system technology that combines microfluidics with BioMEMS or other MEMS devices that explore biological and chemical processes can be used to make devices now commonly known as lab-on-a-chip or micro-total analysis systems (micro-TAS or μTAS). In general, μTAS can be defined as a system capable of performing all sample handling steps together with analytical measurement.

There are many advantages associated with lab-on-a-chip devices. For example, laboratory equipment is scaled down to millimeters. Experiments only require small quantities of reagents or samples; therefore, reducing power consumption and waste. Time of analysis may be significantly reduced. Processes can be automated by integrated electronics, thereby improving performance. Most importantly, cost of operation is significantly reduced. A major challenge and limitation of many lab-on-a-chip and μTAS devices is the unavailability of cost effective packaging schemes that provide a reliable and robust interface from the macro-scale sample and the macro-scale world to the micro-scale devices and sensors on a chip.

The assignee of the present invention has developed a technique to perform cellular growth characterization with electrical impedance measurements at the micro-scale. This technique is described in U.S. Ser. No. 10/837,493, entitled “Apparatus and Method for detecting Live Cells with an Integrated Filter and Growth Detection”, the contents of which are incorporated herein by reference. It would be desirable to augment this technology and other lab-on-a-chip technologies with a cartridge that provides a macro-scale to micro-scale interface. Such a cartridge could simplify sample handling and post-processing decontamination. Such a package should be designed with consideration of material compatibility, reagent/sample delivery system, and cost. It would be highly desirable to provide a packaging platform that performs the biological analysis without user interaction (‘plug and play’) and supports a variety of fluidic networks entering and exiting the biochip.

### SUMMARY OF THE INVENTION

A cartridge includes a first layer to receive a printed circuit board hosting a biochip. A second layer is connected to the first layer and is configured for sample injection to the biochip. The second layer includes a first septum to isolate fluidic ports to the first layer. A third layer is connected to the second layer and includes a fluidic channel configured for fluidic purge operations. The third layer includes a second septum to isolate a fluidic channel from the fourth layer. A fourth layer is connected to the third layer and is configured to receive and route fluidic samples to the third layer.

### BRIEF DESCRIPTION OF THE FIGURES

The invention is more fully appreciated in connection with the following detailed description taken in conjunction with the accompanying drawings, in which:

FIG. 1 is a top perspective exploded view of a BioMEMS cartridge configured in accordance with an embodiment of the invention.

FIG. 2 is a bottom perspective exploded view of the BioMEMS cartridge of FIG. 1.

FIG. 2A is a bottom perspective view of the third layer of the cartridge of FIG. 1.

FIG. 3 is a top perspective exploded view of an injection molded cartridge configured in accordance with an embodiment of the invention.

FIG. 4 is a bottom perspective exploded view of the injection molded cartridge of FIG. 3.

FIG. 5 is a perspective view of a top mold that may be used to form the device of FIG. 3.

FIG. 6 is a perspective view of a bottom mold that may be used to form the device of FIG. 3.

FIG. 7 is an exploded view of a stereolithographic cartridge formed in accordance with an embodiment of the invention.

FIG. 8 is a top view of the cartridge of FIG. 7.

FIG. 9 is a side view of the cartridge of FIG. 8.

FIG. 10 is an enlarged view of a portion of the cartridge of FIG. 9.

FIG. 11 is an exploded view of a cartridge machined in accordance with an embodiment of the invention.

FIG. 12 is a bottom perspective view of the cartridge top of FIG. 11.

FIG. 13 is a top view of the cartridge of FIG. 11.

FIG. 14 is a side view of the cartridge of FIG. 13.

FIG. 15 is an enlarged view of a portion of the cartridge of FIG. 14.

FIG. 16 is a view of the cartridge of FIG. 11 and an associated housing assembly.

FIG. 17 is an exploded view of a fluidic connector that may be used with the device of FIG. 16.

FIG. 18 is the assembled fluidic connector of FIG. 17.

FIG. 19 is a front sectional view of a cartridge of the invention.

FIG. 20 is a side sectional view of the cartridge of FIG. 19.

FIG. 21 is a fluid delivery assembly configured in accordance with an embodiment of the invention.

FIG. 22 illustrates fluidic routing devices that may be used in accordance with an embodiment of the invention.

FIG. 23 illustrates the fluid deliver assembly of FIG. 21 operative with a cartridge of the invention.

FIG. 24 illustrates a mechanized fluid delivery system that may be used with a cartridge of the invention.

Like reference numerals refer to corresponding parts throughout the several views of the drawings.

### DETAILED DESCRIPTION OF THE INVENTION

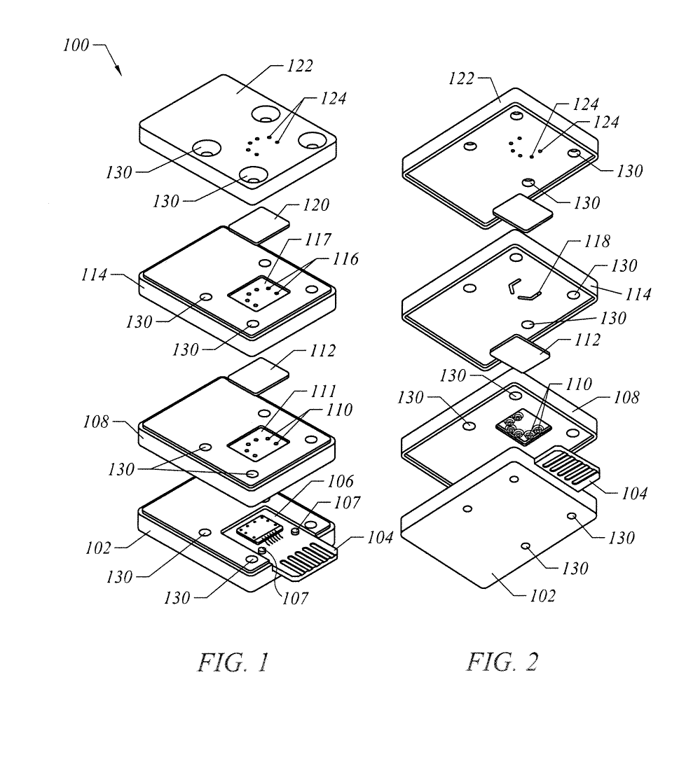

FIG. 1 is an exploded top perspective view of a four layer cartridge 100 constructed in accordance with an embodiment of the invention. The cartridge 100 includes a bottom layer 102 that houses a printed circuit board (PCB) 104, which hosts a biochip (e.g., a BioMEMS chip) 106. PCB alignment pins 107 facilitate correct positioning of the PCB 104 with the biochip 106.

A second layer 108 includes nozzles 110, which attach to fluidic ports of the biochip 106. A septum 112 operates to isolate bypass channels from the nozzles 110.

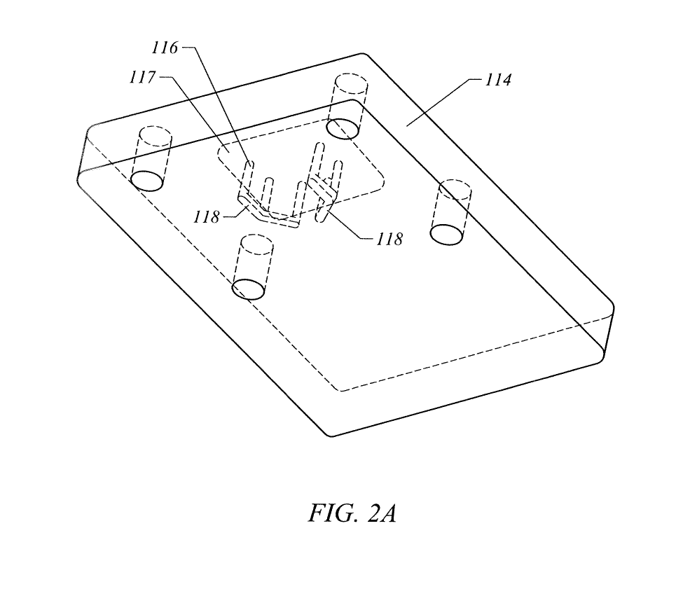

A third layer 114 has channel ports 116, which are aligned with nozzles 110. As shown in FIG. 2, which is an exploded bottom perspective view of the cartridge 100, the third layer 114 has one or more channels 118 that may be used to disinfect needles, as discussed below.

As shown in FIG. 2, a septum 120 rests within a cavity 117 on the third layer 114 and operates to isolate one or more channels. FIG. 2A is an enlarged view of the third layer 114, showing channels 118, cavity 117 and channel ports 116. The figure illustrates that since multiple channel ports 116 share a single channel, a needle in one channel port may be treated (e.g., sterilized) by a fluid injected from another channel port sharing the same channel, as discussed below.

A top layer 122 is positioned over the third layer 116. The top layer 122 includes nozzles 124, which are aligned with nozzles 116 and 110. Fastener apertures 130 are included in the top layer, which align with corresponding apertures 130 on the lower layers. In an alternate embodiment, an adhesive is used to connect the various layers and therefore the apertures 130 are omitted. As discussed below, needles are selectively pressed through the apertures to implement cell concentration/separation, sterilization, and recovery modes of operation.

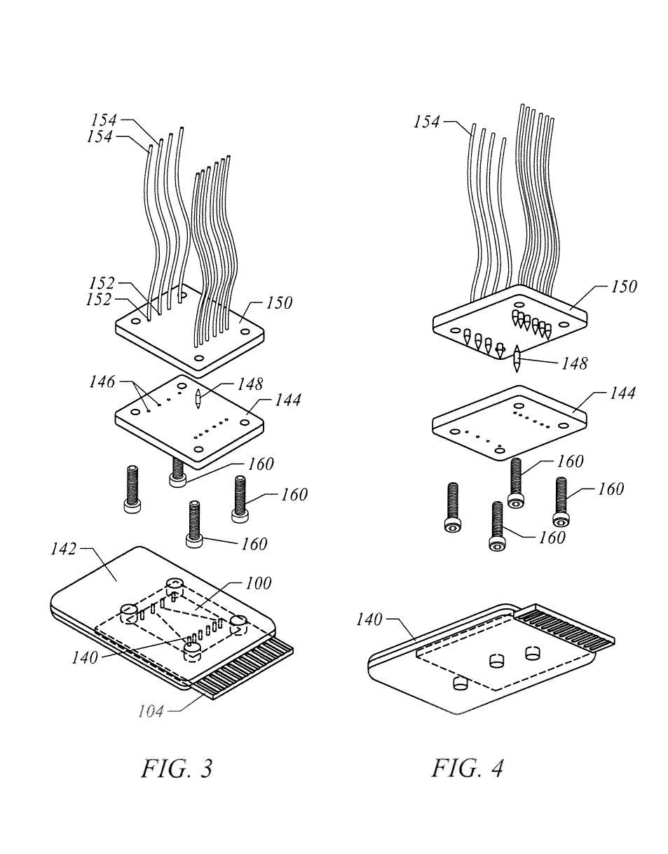

FIG. 3 is an exploded top perspective view of an injection molded housing and associated fluid delivery assembly formed in accordance with an embodiment of the invention. A PCB 104 hosts a biochip 106. Fluidic ports 140 deliver fluids to the biochip 106. The PCB 104 and biochip 106 are encased in an injection molded plastic housing 142. A fluidic connector bottom plate 144 is positioned on the injection molded plastic housing 142. The fluidic connector bottom plate 144 includes apertures 146, which may receive ferrules 148. A fluidic connector top plate 140 is positioned on the fluidic connector bottom plate 144. Apertures 152 are configured to receive microbore tubing 154. Fasteners (e.g., screws) 160 may be used to fix the components together. An exploded bottom perspective view of the components of FIG. 3 is provided in FIG. 4.

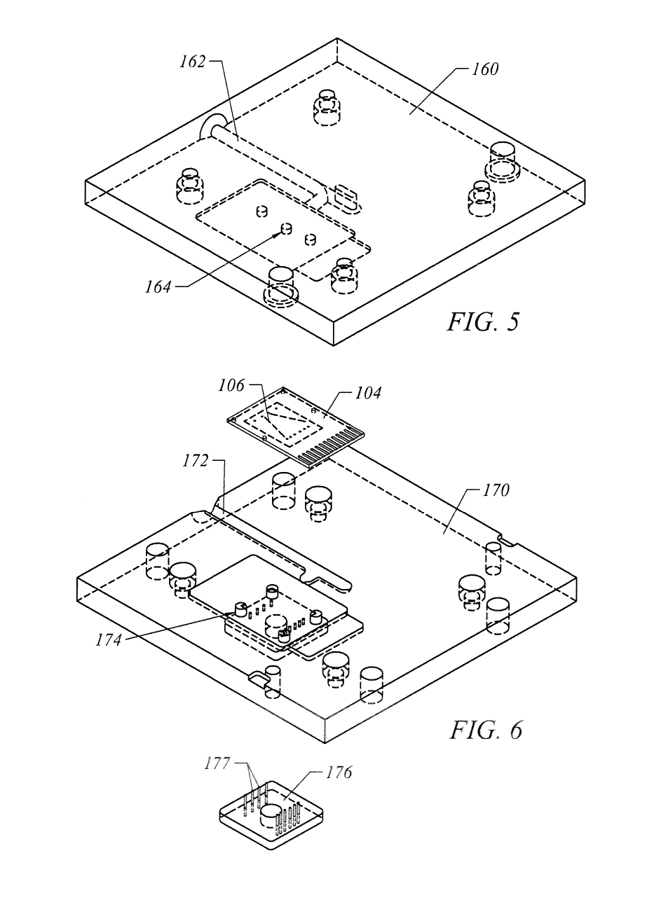

FIG. 5 illustrates a top mold cavity 160, which may be used to form the injection molded plastic 142. The top mold cavity 160 includes an injection channel 162 and at least one PCB support post 164. FIG. 6 illustrates a bottom mold cavity 170 which is operative with the top mold cavity 160. The bottom mold cavity 170 includes an injection channel 172 and alignment structures 174 for the PCB 104. A pin plate 176 with associated pins 177 may be used to form fluidic channels.

The PCB 104 and biochip 106 are placed in the mold cavity with alignment features that index off the edges of the biochip 106. The pins 177 of the pin plate 176 are inserted through the mold and push up against the biochip 106 with a small load. The pins are aligned with the fluidic ports on the biochip and form small fluidic channels on the cartridge after plastic is injected.

The embodiment of FIGS. 3-6 has many beneficial features. For example, no assembly is required on the cartridge. The injection molding is cost effective and has a high yield. The injection molded device is disposable. Further, numerous materials may be chosen based upon biocompatibility and sterilization parameters. The injection molding techniques may be used to form various layers of the device of FIGS. 1 and 2.

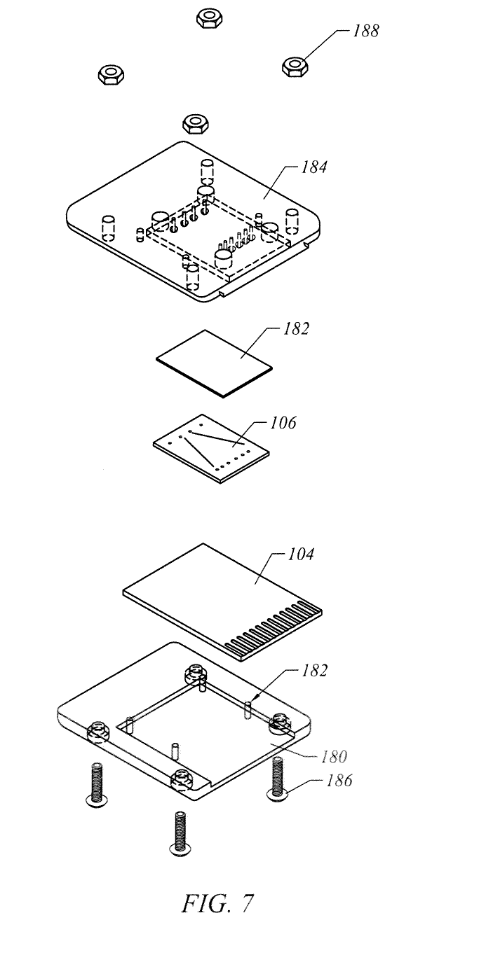

Cartridges of the invention may also be constructed using stereolithography. FIG. 7 illustrates a cartridge formed utilizing stereolithographic techniques. A stereolithograhic bottom layer 180 includes PCB alignment pins 182. The stereolithographic bottom layer 180 receives a PCB 104, which hosts a biochip 106. A gasket 182 is positioned over the biochip 106. A stereolithographic top 184 is positioned over the PCB 104. Screws 186 and nuts 188 may be used to affix the assembly.

Standard stereolithography equipment may be used to fabricate these components. The photosensitive material, SOMOS 11120, may be used to build the cartridge. This material provides smooth surfaces, which can maintain high tolerances for small, critical features. The finished part provides desired properties for handling of the cartridge and alignment features. The gasket 182 (e.g., formed of Teflon), has pre-drilled holes for fluidic passage. The screws 186 provide a seal-tight interface between the cartridge nozzles and the biochip glass.

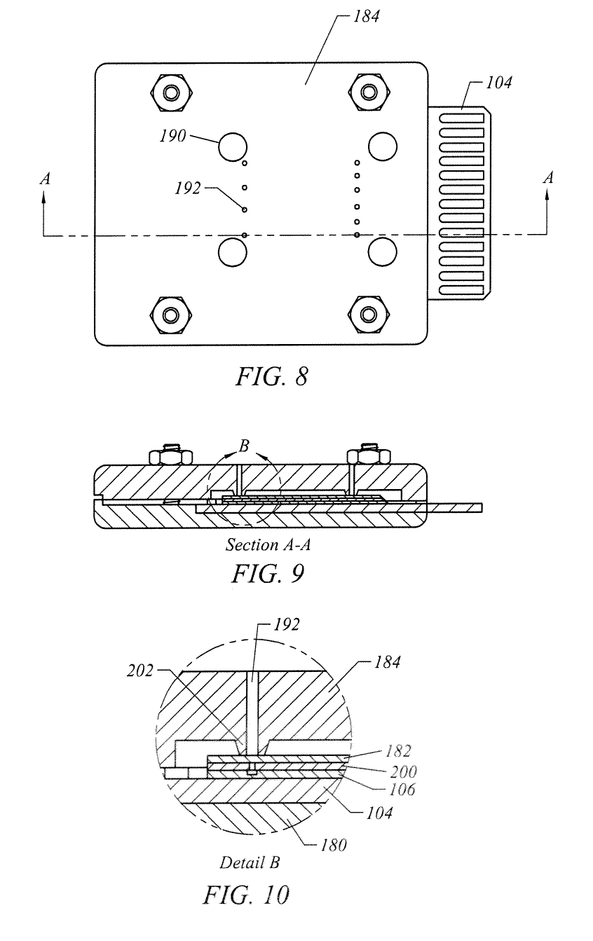

FIG. 8 is a top view of the stereolithographic top 184. The top 184 includes alignment features 190 for external fluidic connectors. The top 184 also includes fluidic ports 192. FIG. 9 is a side view taken along the line A-A of FIG. 8. The round region B of FIG. 9 is enlarged in FIG. 10. FIG. 10 illustrates the stereolithographic bottom 180, the PCB 104, the biochip 106, a glass layer 200, the gasket 182, and the stereolithographic top 184. Fluidic port 192 is formed in the top 184. A nozzle head 202 applies pressure on the gasket 182.

The stereolithographic device allows for a transparent cartridge. It also allows the biochip to be accessible after experimentation. The technique is cost effective and maintains high tolerance.

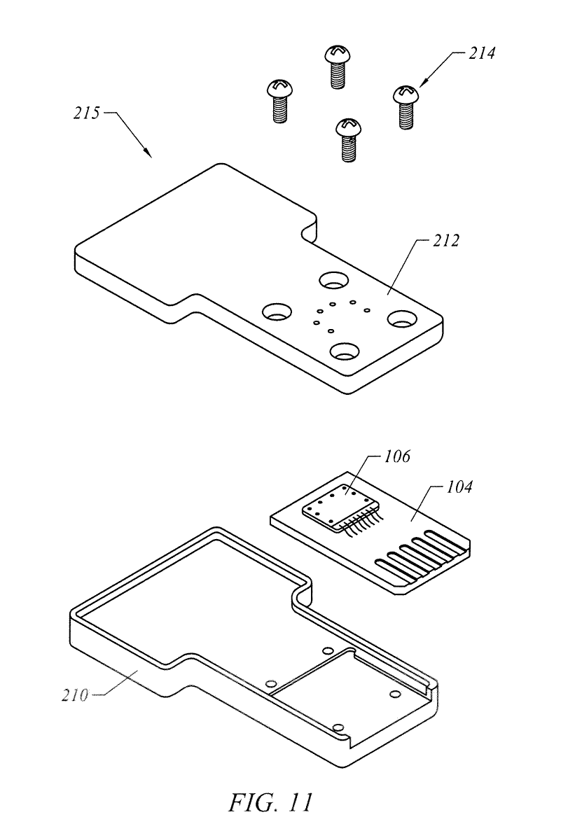

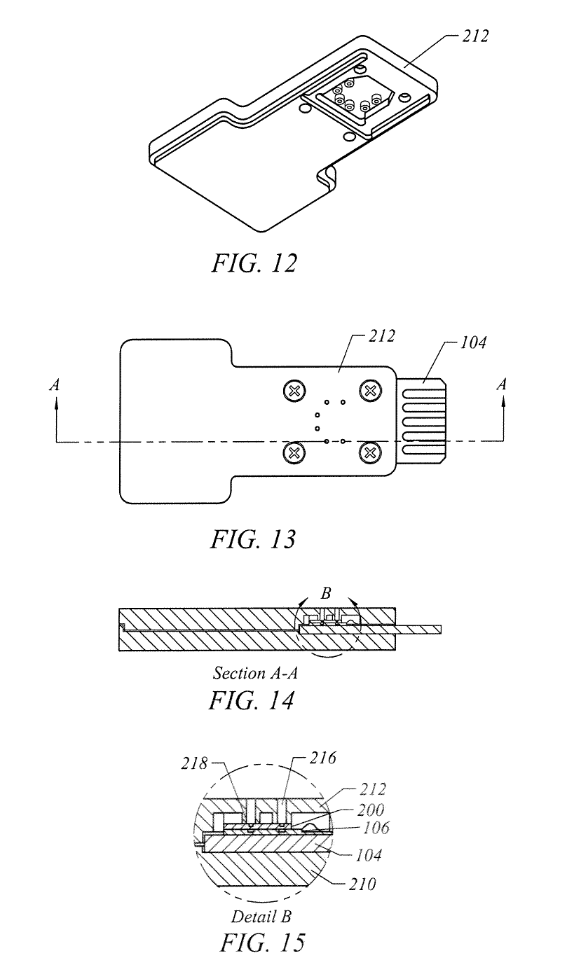

Cartridges of the invention may also be formed using computer numeric control (CNC) machining techniques. In one embodiment, CNC machining is used to form cartridges of Teflon. FIG. 11 illustrates a Teflon base 210, a PCB 104, biochip 106, Teflon top 212, and screws 214 forming a cartridge 215. FIG. 12 is a bottom perspective view of the Teflon top 212. FIG. 13 is a top view of the assembled device of FIG. 11. FIG. 14 is a side view taken along the line A-A of FIG. 13. Circle B of FIG. 14 is enlarged in FIG. 15. FIG. 15 illustrates the Teflon base 210, PCB 104, biochip 106, glass 200, the Teflon top 212, fluidic channels 216 and nozzles 218.

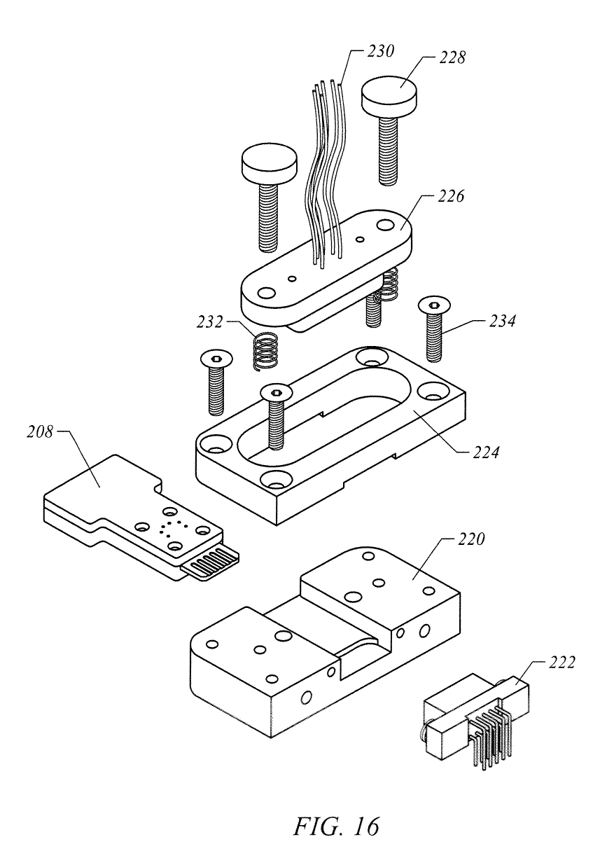

As shown in FIG. 16, the cartridge 215 of FIG. 11 may be positioned in a housing bottom 220, which it interfaces with an electrical connector 222. A housing top 224 is positioned on the housing bottom 220. A fluidic connector 226 is positioned within the housing top 224. The fluidic connector 226 is configured to receive fluidic connector screws 228 and tubing 230. Springs 232 bias the fluidic connector 226 with respect to the housing bottom 220. Screws 234 may be used to affix the assembly.

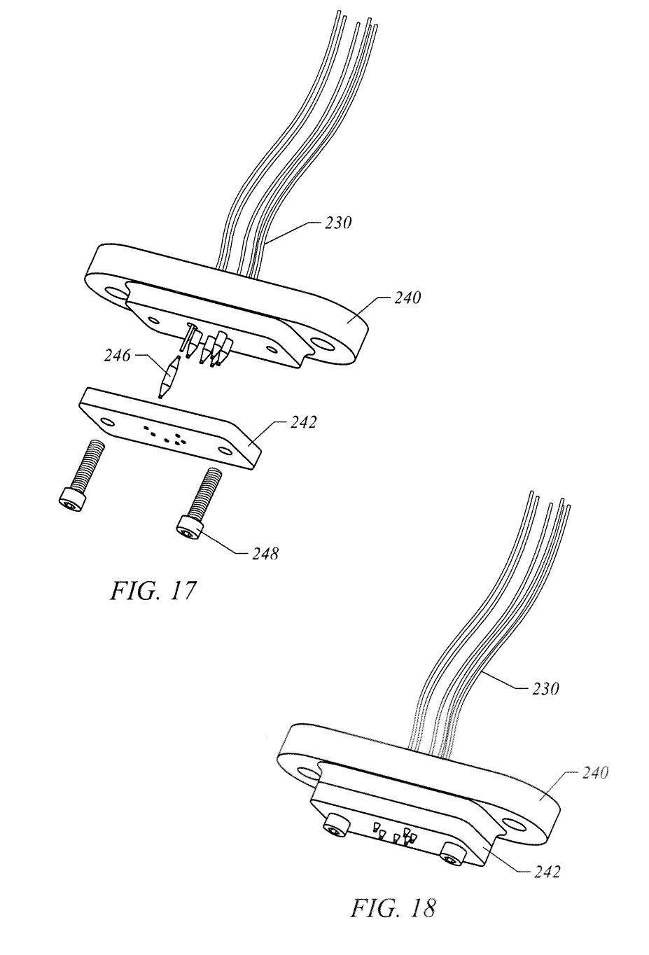

FIG. 17 illustrates an alternate embodiment of a fluidic connector. In particular, the figure illustrates a fluidic connector top 240, which receives tubing 230. A fluidic connector bottom 242 is attachable to the fluidic connector top 240, via screws 248. Ferrules 246 may be positioned within the fluidic connector top 240 and/or fluidic connector bottom 242. FIG. 18 illustrates the assembled device of FIG. 17.

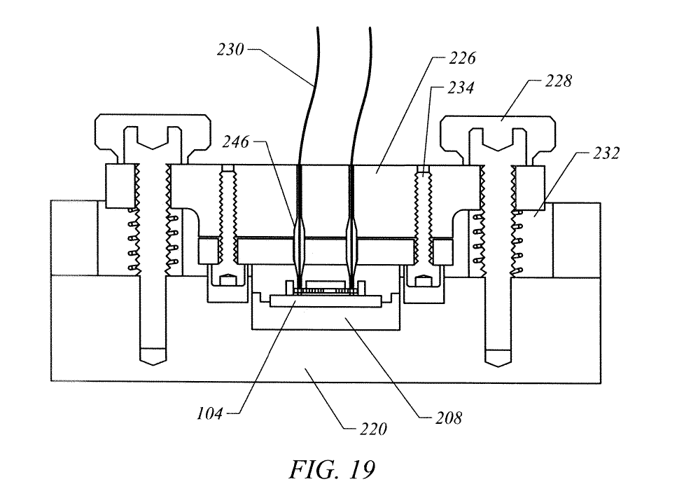

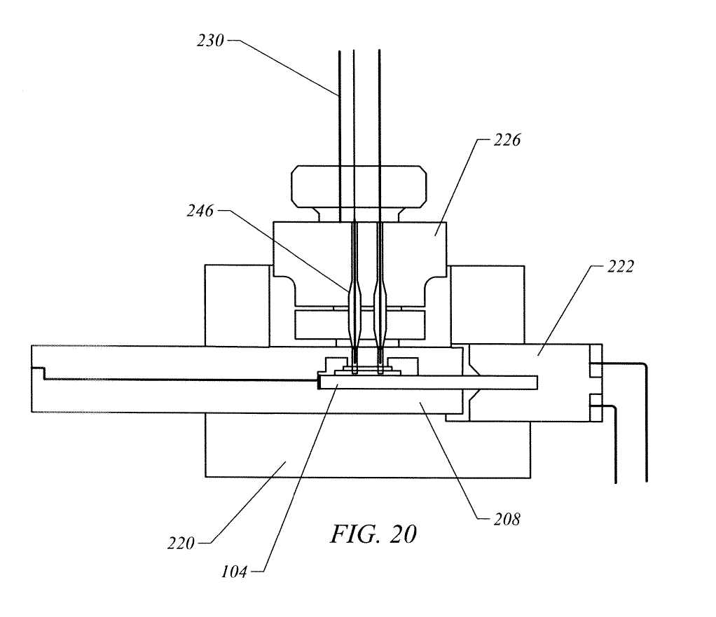

FIG. 19 is a front sectional view of a device formed with the components of FIG. 16 and/or FIG. 17. The figure illustrates a PCB 104 positioned within cartridge 208, which is positioned within housing 220. The figure also illustrates fluidic connector screws 228, screws 234, tubing 230, and ferrules 246. FIG. 20 is a side sectional view of the assembly of FIG. 19.

Various embodiments of the cartridge of the invention have now been disclosed. Attention now turns to details associated with these various embodiments.

The cartridge of the invention is preferably constructed in accordance with the following design criteria:
1. Reduce or eliminate manual tasks of connecting individual fluidic tubes to the biochip.
2. Operate independently from the biochip so that any failure mode on the cartridge might not sacrifice the biochip.
3. Function as a protective shield against physical damage to the biochip and PCB.
4. Provide a leak free seal between each cartridge and biochip on each individual port with pressure up to 150 psi.
5. The biochip does not need to be accessible or visible when the cartridge is assembled.
6. The biochip could be accessible upon completion of experiment for evaluation (e.g., under microscope).
7. The cartridge can encase a PCB except the gold-plated edge connector to accommodate electrical connection.
8. The cartridge does not interfere with any wires or damage any wire bonds between the biochip and PCB.
9. The materials of the cartridge should be inert to most chemicals and be biocompatible. Fluids and bacteria are not absorbed by the material.
10. The physical characteristic of the cartridge remain unchanged when exposed to the conditions developed by the heating pad in the PCB and biochip.
11. The cartridge should be disposable.
12. All ports on the cartridge are sealed during and after test to avoid contamination and to provide safe handling.
13. The cartridge can withstand at least 12 hours of testing.
14. The cartridge design is scalable to accommodate the design changes of the chip.
15. A unit separate from the cartridge delivers fluids via a robust connection to the cartridge. This unit is termed the fluidic connector.
16. The fluidic connector is reusable.
17. Provide a leak free seal between the fluidic connector and cartridge and biochip on each individual port with pressure up to 150 psi.
18. Provide repeatable connections between the fluidic connector and cartridge and biochip with proper alignment.
19. Spillage of any kind of fluids does not occur when the fluidic connector is removed from the cartridge.
20. Any part of the fluidic connector that made contact with fluids during operation needs to be sterilized before the fluidic connector is removed from the cartridge.
21. Ability to remove fluidic connector from cartridge during bacterial growth phase.
22. The fluidic connector can accommodate multiple tests.

The cartridge of the invention may be fabricated from any number of materials. Economically, plastic is an appropriate choice for a disposable cartridge. Plastics can be a base material additive to reinforce certain properties, such as strength, elasticity, opacity, and chemical resistance. At the chemistry level, plastics or polymers are formed by polymerization in which monomers are joined to form linear chains or complex three-dimensional networks of polymer chains. Determined by the molecular structure, polymers can be broadly defined into three classes: thermoplastics (crystalline or non-crystalline), elastomers (rubber), and thermosets (duraplastics). Thermoplastics, which are the most prevalent class of plastics, have either linear or branched polymer chains and can be melted with heat and harden when cooled. Examples of thermoplastics are polyethylene (PE) and polypropylene (PP).

Elastomers are plastics composed of weakly cross-linked polymers and have high resilience characteristics, such as poly(dimethylsiloxane) or PDMS. Thermosets are heavily cross-linked polymers. Unlike thermoplastics and elastomers, thermosets cure and harden irreversibly. If thermosets are overheated, the polymer degrades instead of melts. Typical thermosets are rigid, brittle, and intractable, such as epoxies, alkyd polyester, and urea-formaldehyde plastics.

If the design is a molded part, the polymer should have good flow properties, controllable shrinkage, and good surface finish. The performance of the polymer should also satisfy the assembly process, such as solvent bonding, fasteners, ultrasonic welding, and snap fits. For end use purposes, the plastic of choice needs to accommodate all the physical, mechanical, electrical, biological, and chemical attributes defined in the design criteria. Although the physical, mechanical, and electrical properties are subjectively defined, it is clear that of the material should generally satisfy the handling of the cartridge, proper sealing of liquids, and not affect any signal transmission. However, the biological and chemical attributes require more explanations.

Biocompatibility usually refers to pathological effects on animals. For example, a plastic is considered biocompatible if there are no adverse effects upon implantation; or no release of impurities, extractables, or degradation products. In fact, a global standard has been set forth to regulate a series of tests that would determine if a material is biocompatible; this is documented in ISO 10993, “Biological Evaluation of Medical Devices.” Under this regulation, tests are performed in search for acute, subchronic, and chronic toxicity; irritation to skin, eyes, and mucosal surfaces; sensitization; hemocompatibility; genotoxicity; carcinogenicity; and reproductive effects. In vitro methods, which are formulated under ISO 10993-5, are testing for biologically harmful extractables. Direct contact between the material and mammalian cells are cultured to ensure any adverse effects are nonexistent. For the in vivo method, the FDA has developed a guideline under memorandum G95-1 for testing in the United States. Plastics that meet these specifications will be classified as an USP Class VI biocompatible material. The cartridge should still be safe to handling during all phases of an experiment.

In many embodiments, the cartridge will route three different types of fluids to a biochip: a rinse fluid, a growth fluid, and a sterilizing fluid. The rinse fluid is typically deionized water. The growth media that provides the nutrients for the bacteria usually has a neutral pH with high salt content, such as LB. Of these three types of fluids, the sterilizing fluid is probably the most susceptible to reacting with plastics. At the prototyping stage, the sterilizing solvent is unknown, but the fluid will most likely be an alcohol (e.g., methanol, ethanol, etc.) and it is possible that the solvent could also be a detergent or bleach. Nevertheless, the polymer must maintain its molecular structure and withstand the environment introduced by the sterilizing agents. Tests should be conducted to ensure that the polymer does not craze, crack, discolor, soften, or dissolve.

Based on the foregoing attributes, a cartridge material may be selected from Table I.

TABLE I

| | |
|---|---|
| ABS | Acrylonitrile-Butadiene Styrene |
| PC | Polycarbonate |
| HDPE | High Density Polyethylene |
| PS crystal | Polystyrene crystal |
| PP homo | Polypropylene homopolymer |
| PA T40 | Polyamide T40 |
| POM | Polyoxymethylene (acetal) |
| SMMA | Styrene Methyl Methacrylate |
| COC | Cyclic Olefin Copolymer |
| PCTFE | Polymonochlorotrifluorethylene |
| PVDF | Polyvinylidene Fluoride |
| ETFE | Ethylene Tetrafluoroethylene |
| PPSU | Polyphenylene Sulfone |
| PEI | Polyetherimide |
| PESU | Polyethersulfone |
| PEEK | Polyetheretherketone |
| PTFE | Polytetrafluroethylene |
| FEP | Fluorinated Ethylene Propylene |

Material selection for any given cartridge is a function of mechanical performance, where tensile strength, elasticity, durability, chemical resistance, among others factors are important. In terms of manufacturing, the molding conditions of the plastic, the control of shrinkage, and the workability of the material during assembly are important components that define the quality of the device. The price of the raw material is directly related to the commercial success of the product. The purpose of the cartridge is to be a single use device that can be discarded. Based on these parameters it is important to choose a material that satisfies all the design criteria.

The mold of FIGS. 5 and 6 (and each layer of FIG. 1) may be fabricated using a traditional injection molding process to integrate the micro-fluidic channels directly to the biochip ports. In one embodiment, the sequence of events is as follows. The PCB 104 and biochip 106 are placed in the mold with proper alignment before injecting the plastic. Special features built into the mold will block the open ports in the glass cover and interface directly with the ports on the biochip to create the micro channels. Polymer flows into the mold via channel 162/172, encapsulating the biochip. The assembly can be ejected from the mold and be ready for use.

In this embodiment, the components going into the mold include not only the biochip (silicon and glass), but also the PCB with wire bonding. There are several challenges encountered with the mold design. A key component to the success of the over-mold cartridge is the alignment of the biochip. Misalignment of the features relative to the biochip can result in complete blockage of the micro channels. On a similar front, the design of the features in the mold that interface with the ports on the glass of the biochip is critical to prevent leakage. It is also a concern that the gold wire bonds might be damaged as a result of the molding process due to high temperature (in excess of 150 degrees Celsius) and pressure (in excess of 1200 psi).

Micro-fluidic channels may be formed in the cartridge by hardened steel pins 177 (e.g., with a diameter of 0.032 in.). These pins were planted in a small plate 176 of 6061 Aluminum. By electroplating a small amount of nickel on the pins, a secure press fit is attained. On the underside of the bottom half of the mold, access holes were predrilled matching the pattern of the pins. The block of pins were inserted through the underside of the mold and pressed up against the glass surface of the biochip. In order to maintain pressure on the glass, a spring force is required from the block of pins. A rubber material was placed beneath the block to generate such force.

In one embodiment, the material selection for the mold 160/170 is 7075 Aluminum, which has excellent machining properties and yet the mechanical properties are suitable for a prototyping run. The polymer chosen for the over-mold cartridge design is Dow Plastics' Pellethane 2363-75D polyurethane elastomer, ether based. This polymer is commonly used in healthcare applications at which it is classified biocompatible under USP Class VI. The Pellethane 2363 series has outstanding properties, which include superior resilience, resistance to attack by micro-organism, low extractables, and smooth surfaces. Data has also shown that this polymer is highly resistant to detergent and bleach.

The mold may be implemented with additional posts to hold the biochip-PCB assembly in place. Vent holes may also be placed in the mold to prevent air pockets.

Although the over-mold concept could potential simplify the macro to micro interface, it violates one of the design criterion of accessibility to the biochip after experimentation.

To avoid the high cost associated with mold making, one may fabricate the cartridge of the invention with a rapid prototyping method known as stereolithography (SLA). Making an SLA prototype requires translating a computer model into a .stl file format, which contains ASCII or binary data of triangular surfaces that describe the geometry of the part. Based on these data, the SLA machine will fire a high powered ultra-violet laser into a vat of liquid photosensitive polymer and cure the resin in layers of 0.002-0.010 in. Depending on the size and complexity, a plastic 3-D rendering of the part can be obtained in 1-2 hours. The SLA prototyping method is highly time efficient and will accommodate many design changes without incurring cost of tools and molds.

The design of the SLA cartridge will be different than that of the over-mold cartridge. It is not possible to place the biochip-PCB assembly into the liquid polymer and build the part around it. Hence, the cartridge is built in two halves, as shown in FIG. 7.

The biochip 106 and PCB 104 are housed in between the top and bottom part of the cartridge. The main purpose of the bottom half is to support and provide proper alignment for the biochip-PCB assembly. Since the biochip 106 is on the top side in this design, the alignment can not be achieved using the biochip edges and corners. The epoxy should have full coverage underneath the biochip to ensure even thermal conductivity. As a result, residue of epoxy will form at the edges and corner, which cannot be used as alignment features. Instead, four alignment posts 182 on the bottom half of the cartridge mate with four holes on the PCB. This setup prevents translational motion of the biochip-PCB relative to the cartridge as it engages and disengages from the electrical connector.

The top half of the cartridge has channels in the same configuration as the ports on the biochip. On the bottom side of the top half 184, nozzle features are built on each port that interfaces with the biochip. These nozzles function as pressure concentrators to seal each port. Without the presence of these features, a flat interface could introduce “cross-linking” between adjacent ports and leakage of bacterial samples. Unfortunately, the nozzles cannot function alone due to the hardness of the material. Most photosensitive polymers are thermosets, which are defined as rigid and brittle and cannot soften by heat. The proposed solution was to add an intermediate layer of gasket material 182 to seal the interface between the nozzles on the cartridge and the glass on the biochip. Four gasket materials were selected based on success in macro-scale applications and its potential in microfluidic transport. These materials are Buna-N rubber, PTFE, aramid/SBR, and silicone rubber. The gaskets are in sheet form and have thickness of 0.032 in., except for Buna-N-Rubber, which is 0.062 in. The gasket sheets are cut to the same sizes as the biochip and hole patterns matching that of the ports are drilled with 0.028 in. bit. The following text discusses the relative merits of various gasket materials.

Consistent holes were difficult to obtain with silicone rubber. Buna-N Rubber provides fairly consistent drilled holes and seals well. PTFE material is easy to cut and drill, producing consistent edges and holes. Fluid was successfully passed through, and when the system was disassembled there were no signs of leakage. Aramid/SBR is a material with paper-like characteristics. It is easy to drill, but due to its hardness, it is somewhat difficult to cut. The material may be susceptible to leakage and absorption.

Based on the results of the leakage test, Buna-N rubber and PTFE appear to be the most appropriate gasket material. PTFE is thinner and easier to fabricate; moreover, it is inert to most chemicals and is known to be biocompatible. This gives PTFE a leading edge over Buna-N rubber.

The SLA cartridge prototype is made from DSM Somos WaterShed 11120. This thermoset has exceptionally low water absorption at 0.35%, high modulus of elasticity (2650-2880 MPa), and high transparency. Although WaterShed 11120 can offer unmatched durability, its lack of chemical resistance restricts the cartridge from a complete bacterial incubation.

Before evaluating the next cartridge design, attention turns to the design and functionality of the fluidic connector, the device that makes the macro-to-micro interface possible. The fluidic connector is not a single use component; its purpose is to consistently supply liquids to tightly packed ports on disposable cartridges. Previously, bonded tubes have to be disposed off, along with the biochips, since they are not reusable after bacteria are trapped inside. By isolating the mating tubes through the use of the fluidic connector, the cost of tubing for each test can be conserved through this multi-usage system, and the plug and play capability allows experiments to be conducted on the fly.

A key challenge to the development of the fluidic connector is the compactness of the fluidic ports and the capability to withstand 150 psi of in-line pressure. In order to use a readily available connector with similar capacity, a fanned out channel system needs to be integrated onto the cartridge to deliver fluids in to compact ports.

To overcome the issue of tightly spaced ports, ferrules (e.g., with maximum diameters of 0.062 in.) may be used. FIG. 4 illustrates ferrules that may be used in accordance with an embodiment of the invention.

These ferrules are tapered on both ends and a through hole allows the fitting of a 360 μm diameter tubing. PEEK tubing with 360 μm ID and 150 μm ID were used in one embodiment. Custom designed plates (6061 Aluminum) to fixture the ferrule have patterns identical to that of the ports' locations and also have the same 25 degrees taper as the ferrule. As the plates are brought closer together by the screws, a radial pressure was applied to the tapers of the ferrules, crimping the tubing inside. This secures all the tubes and still allows fluid flow. The tapered portion of the ferrules has different clearance at the bottom than at the top when assembled. The top plate covers most of the tapers as crimping is most effective near the tip. The bottom plate has most of the taper exposed. This allows the nozzle features of the ferrule to protrude through such that it could be used to mate with the cartridge ports (e.g., with a diameter of 0.032 in.). Since the diameter of the ferrule starts at 0.062 in. and tapers at 25 degrees, enough clearance is required to not only allow the nozzles to enter into the port, but to seal around it. When the fluidic connector is assembled, the tubing is attached to the fluidic control system. By compressing the cartridge and the fluidic connector, fluids are successfully delivered.

The effectiveness of the fluidic connector has made testing of the biochip prototypes facile. In fact, the proof of concept on both the cartridge design and fluidic connector has motivated an even more compact biochip with a new bacteria capturing technology. The new capturing mechanism operates through mechanical filters formed by interdigitated channels. By using mechanical filters instead of DEP electrodes, the required surface area of the chip decreases drastically. The size reduces by over 900% with dimensions of 8.650 by 7.175 mm. This enables a minimum port spacing of 2.500 mm.

From the advancement of the biochip, a new set of specifications were developed for a new fluidic connector. The new design will follow the same notion, except the ferrules are more compact. Due to the tight spacing, the 6061 Aluminum plates are not sufficient since bowing occurs before the tubes are securely crimped. Thus, the construction material is replaced with stainless steel 314. The updated design is shown in FIG. 17. This fluidic connector was also updated to work in conjunction with a testing station that houses and aligns the cartridge for incubation. Using this station, the fluidic connector consistently aligns with the ports on the cartridge and two screws can adjust the height of the fluidic connector to control the compression against the cartridge. Upon completion of an experiment, springs disengage the fluidic connector through the adjustment screws.

The success of the fluidic connector has been an integral part to interface macro-to-micro fluidic connections, and the cartridge that is simply the disposable interface between the connector and the biochip has made testing easy and reliable.

Based on the accomplishment associated with the SLA cartridge, the same design philosophy can be followed. The fixture of the PCB and the nozzle interface to the glass of the biochip are necessary for the following design. Although the photopolymer material is incompatible with ethanol, the mechanical design is sensible. Therefore, if the material is substituted with a compatible one and the mechanical design is preserved, a fully functional cartridge can be attained. However, other conventional machining techniques may be employed, such as computer numeric control (CNC). A CNC machined Teflon cartridge was developed to capture the same mechanical performance. Teflon or PTFE have already demonstrated the appropriate sealing properties, and as it turns out, Teflon also has respectable mechanical properties. Although it only has a modulus of elasticity of 0.4-0.8 MPa, its dimensional stability is sufficient for handling of the cartridge. It also has outstanding resistance to water absorption (0-0.01%) due to its hydrophobic characteristics. Teflon is also tolerable to conventional machining (i.e. milling) due to its high resistance to temperature.

A Teflon cartridge may be fabricated via a CNC machine with similar features as the SLA cartridge. FIG. 11 illustrates a machined Teflon cartridge. Cartridges were tested at 100 psi in search of leakage at all interfaces, none were found.

As described in previous sections, the invention includes a technique for delivering fluids into a microfluidic chip with compact port configurations. Up to this point, most of the design specifications have been satisfied to a certain degree. Two design specifications remain a challenge: removable fluidic connector during incubation and the accommodation of multiple tests. The benefit of isolating the fluidic connector from the cartridge during incubation is obvious. Since the population of bacterial cells is increasing, they could potentially find their way into the fluidic control system, which can contaminate future experiments; furthermore, it can be a health hazard. Therefore, the ability to pull out the fluidic connector right after injection and rinse the system with the sterilizing agent minimizes those risks. This also leads to the advantage it has on the next design specification, at which the sterilized fluidic connector can move on to another cartridge and inject another sample for analysis. The option offers the ability to design multi-cartridge systems that can run multiple tests simultaneously.

Isolating the fluidic connector from the cartridge first requires the ports on the cartridge to be sealed from the open environment. This can be accomplished by using silicone rubber to cover the channels. A drawback of this method is the fluidic connector with the ferrule tips is unusable since it does not have the capability to pierce through the rubber. Silicone rubber is actually an ideal material to use to seal off liquids and it is commonly used in the healthcare and medical industry as septum. Vaccines and other liquid medicines are stored in small vials and sealed off from the environment using a septum at the cap. A needle can pierce through to pick up the liquid and the remaining liquid will not spill even after removal of the needle.

To demonstrate the effectiveness of the silicone rubber, a typical laboratory glass vial was filled with DI water. The cap of the vial was drilled to create two holes with diameters of 0.062 in. A 0.032 in. thick silicone rubber sheet (40 Shore A) was cored to the same diameter as the cap, such that when the cap was closed, a tight seal was formed around the perimeter. A 22 gauge stainless steel needle was pierced through one of the holes and then removed. The same needle was then inserted into the other hole and the vial was pressurized with nitrogen at 100 psi. The pressure was allowed to equilibrate inside the vial and dilute soapy water was applied over the previously pierced hole, at the needle-septum interface, and around the perimeter of the cap. The purpose is to identify the location of the leakage, which is indicated where gas bubbles are formed. The end results shows that the pierced septum and the needle-septum interface can withstand at least 100 psi of pressure, and in fact leakage was first observed at the perimeter of the vial when pressure was slightly increased. Curious about the septum's durability, the needle was pierced in each hole twenty more times. Amazingly, the results were the same; no leakage was detected despite the physical distress that was imposed. Although damage to the septum was visible when stretched out it was able to endure the same sealing performance.

Silicone rubber, which is also known as polysiloxane, possesses excellent mechanical properties. This material is compliant under FDA regulations and has been classified as USP Class VI material. Chemical testing was also completed according to requirements, and no adverse effects were found.

Due to silicone rubber's enduring mechanical characteristics and their inertness to biological and chemical components, one can confidently use it to seal off the ports on the cartridge from the environment. Typically, an adhesive is used to secure the seal over the device. A mechanical solution may also be used for quick assembly and immediate usability. The design of the stacking layer cartridge is shown in the schematic of FIGS. 1 and 2.

The base level 102, referred to as Layer 1, has the same design concept as the SLA and CNC machined cartridge containing PCB alignment pins 107 to support and align the biochip-PCB assembly. Layer 1 also contains threads 130 that receive screws that hold the cartridge together. Due to the thread stripping problem faced with Teflon a higher strength material, POM or acetal, may be used. Acetal has higher tensile and flexural modulus, excellent resistant to water absorption, is easy to machine, is compliant with the FDA, and is chemically inert to our standards.

The next component, Layer 2, 108 fits over Layer 1 and the biochip-PCB assembly 104/106. The construction of Layer 2 is similar to previous designs in which cylindrical nozzles were used to interface with the biochip. Teflon remains the material for this layer primarily due to its machinability and sealing properties. The nozzles seal tightly around the ports of the biochip and isolate each port from the others. At the top of Layer 2 a shallow rectangular cavity 111 positions a silicone rubber pad (e.g., 0.032 inches thick) or septum 112.

The next layer, Layer 3 (acetal), 114 compresses the septum 112. This small compression provides the adequate seal on the ports of the cartridge. This method of securing the septum is quick and reliable. There are added advantages with this layered technology of the cartridge. By stacking on more layers, different fluidic activities can be performed independently at each layer. For Layer 3 114, two channels 118 (e.g., with cross-section 1.00×0.79 mm) were milled out on the bottom side connecting inputs to the output. Note that one channel is dedicated for the sample side and the other for the reference side. The manifold that connects the inputs to an output without flow through the biochip has added benefits in terms of fluidic control. It has been determined that the flow is most restrictive at the biochip due the small effective cross section of the channels and filter elements. In one embodiment, the maximum flow rate through the biochip is recorded to be approximately 20 μL/min. In cases where the system needs to switch fluids, all fluid paths leading to the biochip must be purged of the liquid currently in the line. Sometimes the total purging volume is 5-10 times the volume of the entire fluid path. The limited flow rate on the biochip consumes significant time just to switch fluids. Therefore, the bypassing manifold offers a versatile solution of switching liquids swiftly. Although the fluids inside the biochip and the ports of Layer 2 still need to be purged when switching liquids, this volume is considerably less than the fluid lines leading up to it; ˜2 μL versus 2-5 mL. To seal off this purging layer from the outside, the top of Layer 3 114 also has a shallow rectangular cavity 117 for the silicone rubber or septum 120.

Furthermore, these channels 118 allow the rinsing and disinfection of the hypodermic needles used to deliver fluids to the biochip. After delivering a sample to the biochip via Layer 2, the needles are pulled to Layer 3 and disinfectant fluid is flowed through the needles and channels to disinfect the insides and the outsides of the needles that came in touch with the fluid in Layer 2. After such disinfection, the needles can be extracted from the cartridge altogether and are ready for dispensing the next sample.

Layer 4 (acetal) 122, the last layer in this cartridge design, is positioned onto Layer 3 114. Screws squeeze all four layers together forming a complete cartridge. Alternately, the screws may be used to hold adjacent layers together while an adhesive sets, and then the screws may be removed. In one embodiment, the cartridge has the following dimensions: 40.00×30.00×19.09 mm. In one embodiment, from Layer 2 to Layer 4 all the ports have diameters of 0.032 in. Hypodermic needles are used to pierce through the septa in these ports to deliver fluids via Layer 2 or purge via Layer 3. The significance of having Layer 1, 3, and 4 being fabricated out of acetal is for mechanical strength and reusability. From a laboratory prototyping perspective, only Layer 2 needs to be changed from test to test due to permanent compression of Teflon.

The innovation of sealing off the cartridge using silicone rubber septum during incubation with the integrated ability to purge at high flow rate requires a fluid delivery system. A hypodermic needle is a suitable instrument, but the tip style of the needle may influence performance. Two different styles of needles were selected from Hamilton Company (Reno, Nev.) for evaluation. Point Style 2 has the port at the end with the tip beveled, and Point Style 5 has a conical tip and the port on the side. Needles of both styles were punctured into a sheet of silicone rubber and thin wires were fed through the needles to look for signs of coring. For the Point Style 2, small bits of the rubber built up inside the needle, which means that it would require regular maintenance. For Point Style 5, needle coring is nearly impossible and makes it ideal for this application.

Preferably, the actuation of the fluidic connector is automated. The most flexible option is a linear motor that can operate vertically with gravity. The vertical configuration is desirable because the preferable orientation for the biochip is horizontal. A linear motor can be programmed to adjust the height of the needle according to the specification set forth by the cartridge.

To this end, the design of the fluidic connector needs to stay within the limits suggested by the linear motor's operating conditions, and also meet the guidelines specified in the design criteria. The linear motor used with one embodiment of the invention is Parker Hannifin Corporation's MX80S Leadscrew Driven Motorized Stages with travel range of 50 mm. Although the 2.0 mm ballscrew drive system can handle higher payload it is also double the cost of the 2.0 mm leadscrew drive system. Some of the key attributes of the MX80S are +/−5.0 μm repeatability, 45 μm accuracy, and has a thrust load capacity of 44 N (10 lb). The thrust load capacity is an important consideration since the linear actuator will operate at the vertical orientation. This means that the total weight of the carriage mass (194 g) and fluidic connector should be well below 10 lb. Another concern in regard to the thrust load capacity is the force required to puncture the septum with the array of needles on the fluidic connector. To evaluate the amount of required force, the cartridge was placed on a scale and zeroed. Using different gauge needles (24, 26, and 28) and different tip styles (Point Style 2, and Point Style 5) the force to pierce through the first septum and second layer at each port was recorded. The tests indicate that the smaller the needle, the less force it requires, hence the 28 gauge needle is the most effective. However, the bore on the 28 gauge is small (0.0065 in) and could reduce flow rate during purging. The Point Style 2 needle also shows that less effort is required, which is contributed by the sharp beveled tip. However, we know coring causes blockage of flow. One additional property that shows particularly interesting result is the amount of compression on the silicone rubber. It appears that the higher the compression, the more force it requires.

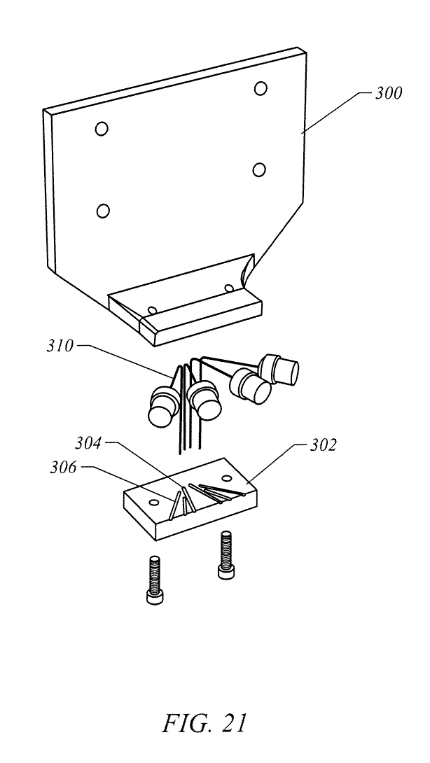

The design of the fluidic connector housing four (capable of six) hypodermic needles is shown in FIG. 21. The fluidic connector mounting plate 300, which has an L-shape, bolts to the carriage of the linear motor and allows the bottom plate 302 to be fastened on. The bottom plate 302 has holes 304 that index the hypodermic needles and precisely machined grooves 306 contour the bend radius of each needle. The groves 306 are then fanned out to accommodate the needle's hub spacing. By putting together the bottom plate to the mounting plate with 90 degrees bent needles 310, the vertical force can be transferred to the tip of the needles.

Alignment holes for the needle just above the cartridge are used. If improperly aligned, the needle could miss the port on the cartridge and cause damages to the fluidic connector and potentially the linear motor. The linear motor has been programmed and the testing of the biochip is automated. Purge and needle disinfection cycles can be done by simply adjusting the tips of the needles to Layer 3 of the cartridge. Sample injection of fluids to the biochip will occur by moving the needle tips to Layer 2. Once incubation starts, the system can be sterilized by flowing through alcohol with needle tips in Layer 3. The fluidic connector can then be removed and perform functions on other cartridges.

Techniques for delivering reagents and samples to the biochip have been described. The samples and reagents may now be processed in accordance with the techniques described in the previously identified patent application, U.S. Ser. No. 10/837,493, entitled “Apparatus and Method for detecting Live Cells with an Integrated Filter and Growth Detection”.

A dilute concentration of contaminants, on the order of 10 to 100 cfu/mL, could potentially cause detrimental effects. It could take days before obtaining any conclusive results if traditional growth and culturing techniques are used. To alleviate this problem, the bacterial cells can be gathered and concentrated into a smaller volume for quicker detection. The following discussion addresses the issue of cell concentration and recovery (CCR). Implementation of a CCR system is a vital step to reduce the time of detection. Without the CCR system, detecting pathogenic microorganisms with the biochip could take just as long as traditional culturing methods. Flow resistance through the biochip is the limiting factor. Unfortunately, the biochip can only handle a maximum flow rate of 20 μL/min, with 15 μL/min being typical, and therefore more than 80 hours is required to complete the filtration of 100 mL of sample. This length of time is unacceptable and it is necessary to reduce the volume introduced into the biochip in order to preserve time. The purpose of the CCR is exactly that. The CCR system should couple with the biochip, take the 100 mL sample and filter out the fluid in a matter of minutes and capture the bacteria in a volume of 50-100 μL. This reduced volume containing the microorganism will be delivered into the biochip, and the total time for the fluid transport is within 20 minutes as opposed to 80 hours.

Commercially, there are many types of filters that can separate particles the size of bacterium from their suspending fluids. However, we are not only interested in separation but also in recovering the filtrates. One traditional type of filter that has proven to be effective in both separation/concentration and recovery is the flat-sheet membrane filter. An alternative to this is the hollow fiber. Currently, most applications that utilize hollow fibers are configured for tangential flow or cross flow, which are common for separation only. In this application, flow will be introduced through the cavity of the hollow fiber and operate under a dead-end mode for concentration and recovery. There are several advantages to operating under these conditions. First, by employing hollow fibers for filtration, higher surface area to volume ratio is achieved; thus, the concentrated volume can be even smaller than that of membrane filters. Second, by blocking the end of the hollow fiber under dead-end mode there will be a higher flux through the permeate due to higher trans-membrane pressure. Third, due to the ease to integration using commercially available fittings and manifolds, the concentration and recovery process can be easily adapted for automation.

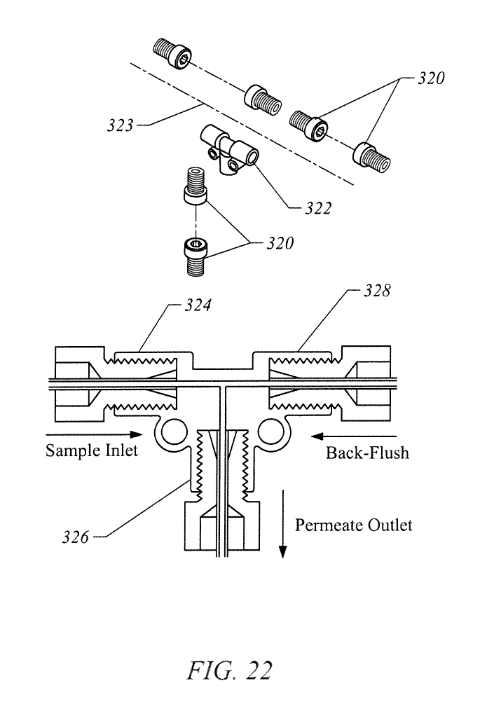

FIG. 22 illustrates a CCR system that may be used in conjunction with the invention. In this embodiment, the CCR is composed of sections of 1/16 in OD polymeric tubing 320 with ID of 0.040 in that can accommodate up to two hollow fibers 323. The hollow fibers 323 are fabricated from polysulfone with nominal pore size of 0.22 μm and have OD and ID of 360 μm and 280 μm, respectively. At each end of the hollow fibers the two segments of tubing are bonded via epoxy adhesive. It has been determined that if the hollow fiber protrudes beyond the end of the tubing and epoxy is applied such that it wicks between the inner wall of the 1/16 in tubing and the outer wall of the hollow fiber, the adhesive will not seep through the pores and clog the cavity of the hollow fiber; this technique is known as potting. Once the adhesive is cured, a razor blade is used to trim off the ends to create the inlet and outlet of the CCR system. The two unpotted ends of the tubing in the mid-section of the hollow fiber are conjoined by a tee 322 that acts as a manifold for the permeate outlet.

The CCR system that has been established contains an inlet 324 for sample loading, a permeate 326 for outlet of filtered fluids, and a retentate 328 that operates either in dead-end mode or back-flush mode for recovery. Prior to sample loading, preparation of the bacterial cells was done using a phosphate buffered saline (PBS) buffer. Traditional plate culture was performed on either Luria borth (LB) or brain heart infusion (BHI) to confirm the cell count between 10 to 100 cfu/mL. During injection of the sample, the valve at the retentate is close to initiate the dead-end mode. The sample is introduced at a rate up to 5 mL/min depending on the number of hollow fibers in the CCR. Bacteria within the sample will begin to collect inside the cavity of the hollow fibers and the remaining fluids should be passed through the permeate. After the sample volume is passed through, the valve at the retentate will open to allow flow to enter for back-flushing. The fluid flow back flushed through the permeate will release the bacteria trapped on top of the pores. The total back-flush volume was 100 μL. This recovered concentrated volume is then cultured in order to determine the recovery rate. The average rate of recovery ranges from 50-70%. The extracted volume from the permeate was also plated to ensure the integrity of the membrane construction and no growth was observed in the permeate fluid.

Since the bacteria will travel along the same path as the fluid, it is expected that the number of cells will slowly decrease along the hollow fiber. This is actually beneficial during the recovery process because the bacterial cells are grouped together, and thus there is less adhesion to the membrane wall. Potentially, a higher recovery rate can be achieved for the same back-flush volume. It has also been determined that a hollow fiber's length of 10 cm appears to be adequate for flow rate around 5 mL/min. The number of hollow fibers to include in the CCR system depends on the initial concentration of the sample. The area of a bacterial monolayer should be less than ⅔ of the inner area of the hollow fiber. Additional hollow fibers should be added if the monolayer exceeds this requirement. Presumably, if the initial concentration is low and the CCR system contains more than one hollow fiber, the recovery rate should not be affected. Bacterial cells collect near the inlets with even distribution among the hollow fibers.

As demonstrated in the computer model and in laboratory testing, the majority of the cells gather in the vicinity of the inlet. Thus, using the back-flush method is more effective and recovery rate improves. The results from the simulation and the experiment provide valuable information in regards to design criteria of a CCR cartridge system.

The invention provides an economical and reliable packaging scheme that interfaces macro-scale fluid samples to microfluidics for chip level sensing of biological agents. There has been considerable amount of research conducted in the field of BioMEMS recently. There is no doubt that BioMEMS will innovate healthcare, defense, and many other areas. However, there is an unbalanced amount of research conducted between the BioMEMS device development and the packaging/interfacing sector. Impractical packaging or interfacing often times lead to inconsistent results and wasted time. Hence, commercial products that integrate BioMEMS are rarely seen. This invention relates to the design and fabrication of a highly functional cartridge that interfaces with a microfluidic biochip.

The cartridge designs eliminate the tedious and time consuming task of tube bonding using epoxy and make validation of the biochip more robust and reliable. Due to the added functionality of the cartridge, a list of design criteria was set forth for the packaging specifications. Although performance is important, safety is also a major concern. The sample for analysis could potentially be pathogenic. The cartridge has to hermetically seal the biochip away from the environment during all phases of the process for safety and sterility. In order to demonstrate an effective and inexpensive solution, several manufacturing methods were explored with polymer being the primary choice of material. We have examined the feasibility of injection molding plastic over the entire biochip assembly, the use of photopolymer material from SLA rapid prototyping method, and computer numeric control machining (CNC) on workable material such as PTFE (Teflon) and acetal (Delrin). Thorough evaluation of these techniques led to the final design of a CNC machined cartridge with multi-layers construction that is capable of hermetically sealing off the biochip from the environment. The multi-layers cartridge also provided a purge point without the flow resistance imposed by the biochip, which also allowed rapid switching of fluids delivered into the biochip.

The cell concentration and recovery (CCR) unit is a key component in supporting rapid detection of microorganisms on the biochip. The CCR system has demonstrated its ability to concentrate a dilute volume of 10-100 cfu/mL in 100 mL and recovered most of the bacteria in a volume of 100 μL. The success of the CCR system was through the choice of filter mechanism. In this case, a hollow fiber with nominal pore size of 0.22 μm was used. Flow was introduced through the cavity of the hollow fiber with its end blocked. This mode of operation has a high surface to volume ratio and high flux through the membrane. A simulation using a Fluent, a computational fluid dynamic (CFD) software, and a qualitative experiment using filter papers was employed to characterize the flow inside and outside of the hollow fiber. Both validations were consistent with one another and concluded that back-flushing from the retentate is most suitable since the majority of bacterial cells are collected near the inlet of the hollow fiber.

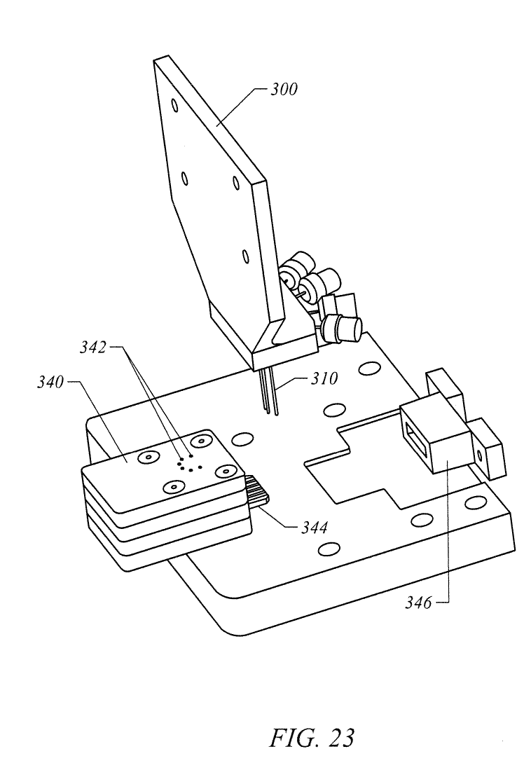

FIG. 23 illustrates the connector mounting plate 300 of FIG. 21 positioned relative to a multi-layer cartridge 340 of the invention. The needles 310 are selectively inserted into the cartridge apertures 342. The figure also illustrates an electrical connection pads 344 of PCB 104 that interfaces with a socket 346.

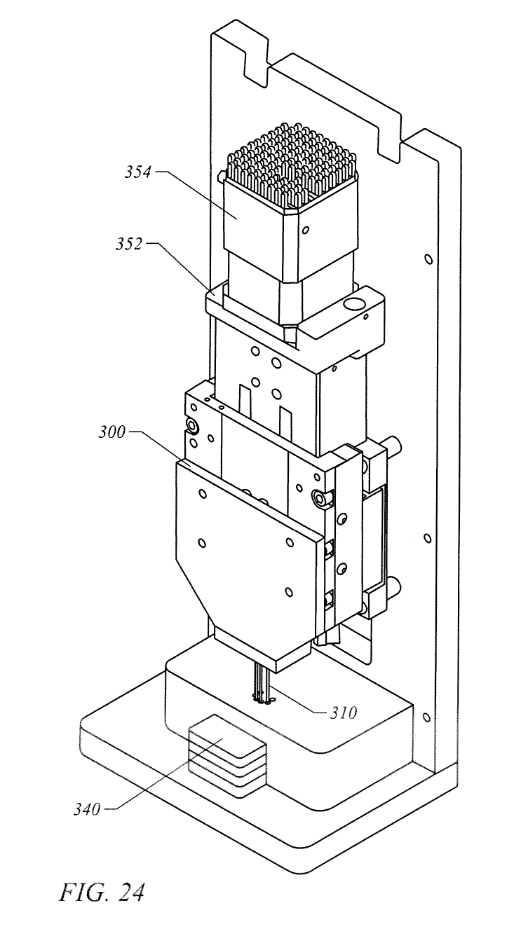

FIG. 24 illustrates the connector mounting plate 300 attached to a slide 352, which is controlled by a motor 354, allowing needles 310 to be positioned at different layers of the multi-layer cartridge 340.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to best explain the principles of the invention and its practical applications, they thereby enable others skilled in the art to best utilize the invention and various embodiments with various modifications as are suited to the particular use contemplated. It is intended that the following claims and their equivalents define the scope of the invention.

1. A cartridge, comprising:
a first layer to receive a printed circuit board hosting a biochip;
a second layer, connected to the first layer, configured for sample injection to the biochip, the second layer including a first septum to isolate fluidic ports;
a third layer, connected to the second layer, configured for fluidic purge operations, the third layer including a second septum to isolate a fluidic channel; and
a fourth layer, connected to the third layer, configured to receive and route fluidic samples to the third layer.

2. The cartridge of claim 1 wherein at least one layer is formed of Teflon.

3. The cartridge of claim 1 wherein at least one layer is formed of Acetal.

4. The cartridge of claim 1 wherein at least one layer is formed of a polymer.

5. The cartridge of claim 1 wherein at least one septum is formed of polysiloxane.

6. The cartridge of claim 1 wherein the fourth layer is configured to receive fluidic samples from a needle.

7. The cartridge of claim 6 wherein the needle is sterilized at the third layer.

8. The cartridge of claim 1 wherein a plurality of needles are utilized to access different layers.

9. The cartridge of claim 1 configured to process a rinse fluid, a growth fluid, and a sterilizing fluid.

10. The cartridge of claim 9 wherein the rinse fluid, the growth fluid and the sterilizing fluid are received from outside the cartridge.

11. The cartridge of claim 1 wherein the first layer contains printed circuit board alignment pins to facilitate functional positioning of the biochip.

12. The cartridge of claim 1 wherein the second layer contains nozzles that make functional contact with fluid ports of the biochip and isolate the fluid ports from each other.

13. The cartridge of claim 1 wherein the third layer contains fluid channels with multiple inputs.

14. A disposable cartridge, comprising:
a first layer to receive a printed circuit board hosting a biochip, the first layer including alignments pins to position the printed circuit board;
a second layer, connected to the first layer, with sealing nozzles configured for sample injection to the biochip, the second layer including a first septum to isolate fluidic ports;
a third layer, connected to the second layer, configured with channels for fluidic purge and needle disinfection operations, the third layer including a second septum to isolate the channels; and
a fourth layer, connected to the third layer, configured to receive and route fluidic samples to the third layer.

15. The cartridge of claim 14 wherein a plurality of needles are utilized to access different layers.

16. The cartridge of claim 15 wherein the needles are positioned with dispensing tips within the second layer to inject fluid into the biochip.

17. The cartridge of claim 15 wherein the needles are positioned with dispensing tips within the third layer to purge fluid paths.

18. The cartridge of claim 15 wherein the needles are positioned with dispensing tips within the third layer to disinfect the dispensing tips.

19. The cartridge of claim 14 configured to process a rinse fluid, a growth fluid, and a sterilizing fluid.

20. The cartridge of claim 19 wherein the rinse fluid, the growth fluid and the sterilizing fluid are received from outside the cartridge.
